(19) United States
(12) Patent Application Publication
(10) Pub. No.: US 2008/0147149 A1
(43) Pub. Date: Jun. 19, 2008

(54) Device and method for stabilization of eye during eye surgery

(76) Inventor: Jaswant Singh Pannu, Coral Springs, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 11/638,923

(22) Filed: Dec. 13, 2006

(51) Int. Cl. A61F 9/00

(52) U.S. Cl. 607/91

(57) ABSTRACT

An eye-fixation device is provided having an eyecup and a source of light within the eyecup. The device can further include an eyecup fixation element, a power supply, and electric, fiber-optic or chemical light sources. A control circuit can cyclically vary the intensity of the light produced by the electric light source. In a method of using the device to reduce motion of an eye during a medical procedure, an eye is selected for treatment and an eyecup is placed over the eye that is not being treated. Light is then introduced into the eyecup.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a device and method for use in medical procedures, and more particularly a device and method for inhibiting motion of an eye.

### BACKGROUND OF THE INVENTION

Medical procedures performed on the eye, such as corrective vision or cataract surgery, are almost always performed while a patient is fully awake or medicated, but conscious. During the procedure, the patient is often asked to try to not move the eye being treated. However, almost all patients find compliance with this request difficult. While it is possible to hold an eye steady with forceps, clamps and the like, doing so is highly undesirable as such contact can distort the shape of the eye and/or increase pressure within the eye leading to an unsatisfactory procedural result. Additionally, mechanical fixation of the eye causes discomfort for the patient and manipulation of the grasping or fixation device can occupy one of the doctor's hands. Thus, it would be desirable to provide a method and/or device to assist the patient in comfortably holding the eye still without physically contacting it that also does not occupy one of the doctor's hands.

### SUMMARY OF THE INVENTION

The present invention advantageously provides a method and system for inhibiting motion of an eye. In an exemplary embodiment, an eye-fixation device is provided having an eyecup and a source of light within the eyecup. The device can further include an eyecup fixation element, a power supply, and electric, fiber-optic or chemical light sources. A control circuit can cyclically vary the intensity of the light produced by the electric light source.

In an exemplary method of using the device to reduce motion of an eye during a medical procedure, an eye is selected for treatment and an eyecup is placed over the eye that is not being treated. Light is then directed to the eye from a light source within, integral with, or external to the eyecup.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of the exterior of an eye-fixation device in accordance with the invention;

FIG. 2 is a perspective view of the interior of the eye-fixation device of FIG. 1.

FIG. 3 is a plan view of the interior of the eye-fixation device shown in FIGS. 1 and 2

FIG. 4 illustrates the eye-fixation device of FIGS. 1-3 provided with an exemplary securing strap;

FIG. 5 is a sectional view of an eye-fixation device in accordance with the invention showing an electric light source;

FIG. 6 is a sectional view of an eye-fixation device in accordance with the invention depicting an alternative configuration for an electric light source; and

FIG. 7 is a sectional view of an eye-fixation device in accordance with the invention depicting yet another light source configuration;

FIG. 8 illustrates that eye-fixation device in accordance with the invention have multiple light sources; and

FIG. 9 illustrates placement of the eye-fixation device on a patient's right eye in preparation for treatment of the left eye.

### DETAILED DESCRIPTION OF THE INVENTION

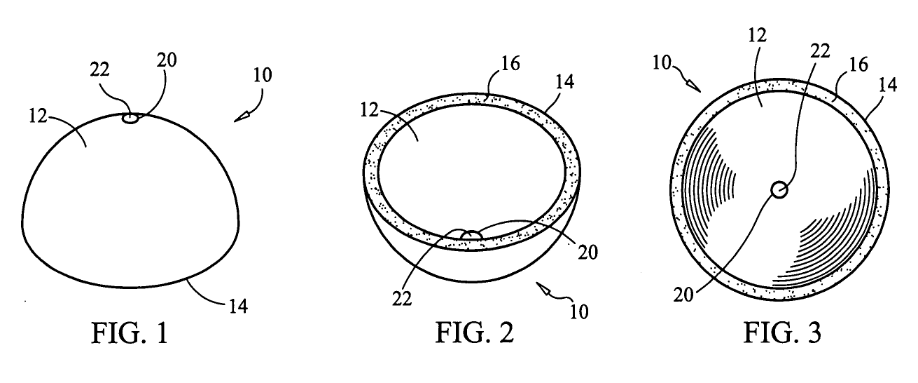

FIGS. 1, 2 and 3 depict an eye-fixation device in accordance with the invention from various perspectives. The eye-fixation device includes an eyecup 10 having a body portion 12 and a lip 14. In exemplary embodiments, the eyecup can be any shape to include circular, oval, oblong, rectangular, irregular, etc. Generally, the particular size and shape of the eyecup 10 is not of particular importance. However, the eyecup 10 should have sufficient circumference so that the lip 14 of the eyecup can be placed on the skin around the eye or on the face and not on the eye itself. In an exemplary eyecup 10, the dimensions are such that the lip 14 rests on or near the eyebrow, the cheek, the temple and the side of the nose. The lip 14 can be very soft and compliant foam, whereas the body portion may be less flexible. Similarly, the interior of the eyecup 10 should be configured so that the surface of the eye, eyelashes, and eyelids, will not be contacted by the body 12 of the eyecup or other elements of the device.

As shown most clearly in FIG. 2, the interior of the body 12 can be lofted or provided with a concavity. In an exemplary device, the interior of the body is lofted so that the distal region of the eyecup (with respect to the eye) is between about two and twenty centimeters from the surface of the eye.

The eyecup 10 can further include an eyecup fixation element, such as a medical adhesive 16 applied to all or a portion of the lip 14. Although fixation of the eyecup 12 can simply be provided by the weight of the eyecup itself on a recumbent patient, or by a weighted ring or strip of fabric, simple adhesive fixation is adequate and less expensive. Alternatively, adhesive tape (not shown) can be used to secure the eyecup 12 to a patient.

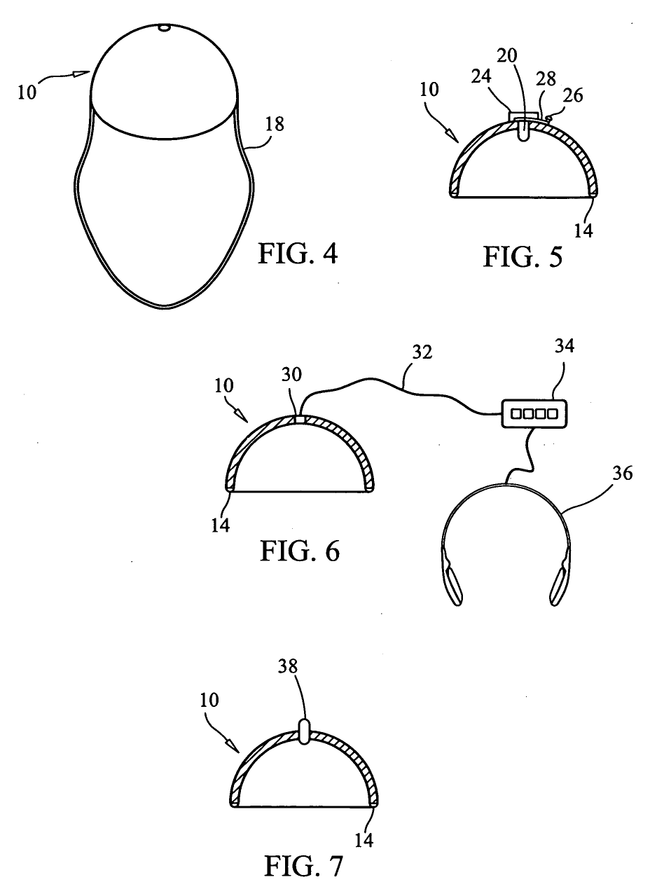

Yet another fixation element is shown in FIG. 4, wherein the eyecup fixation element includes a strap 18 secured to the eyecup 10. All or a portion of the strap 18 can include elastic material. Thusly equipped, the eyecup 10 can be secured over a patient's eye (as shown in FIG. 9) like a protective eye patch. However, unlike a protective eye patch, the eyecup 10 of the present invention is secured over and around an eye such that the eye is placed in relative darkness but for the light provided by the eyecup as described hereinafter.

Continuing to refer to FIGS. 1 and 3, a source of light 20 is provided within the eyecup 10 so that light impinges directly upon the eye. As shown, the light source 20 can include a small aperture in the body 12 of the eyecup 10 through which light outside the eyecup may enter. A transparent or translucent element 22 can span the aperture to both protect the eye from physical objects that might pass through the aperture. Additionally, the element 22 can diffuse, concentrate, or filter light that passes through the element.

Referring now to FIG. 5, an eyecup (shown in a sectional view), such as illustrated in FIGS. 1 and 2, is provided with an electric light source 20. The electric light source can include an incandescent bulb or a light emitting diode (LED), or other electric lamp or light source as is known in the art, that shines through the aperture or that is extended in whole or in part through the aperture so as to be able to direct light within the eyecup 10. A power supply is in communication with the electric light source 20. As shown in FIG. 3, the power supply can be a battery 24, such as a small and light watch or hearing aid battery securable to the eyecup 10. The battery can be removable for reuse, if desired. A control element, such as a switch 26 can be placed in communication with the battery 24 and the electric light source 20 to control power flow to the electric light source. Alternatively, the electric light source 20 can be illuminated when the battery 24 is mated to the eyecup 10. Further, in addition to a switch 26, or in lieu thereof, a control circuit 28 can be provided in communication with the electric light source 20 and the power supply to control the intensity of the light produced by the electric light source and/or to cyclically vary the intensity of the light produced by the electric light source (e.g., to make the light blink or flash “on” and “off”).

Further with regard to the electric light source, the color of the light emitted can be selected for both patient comfort and ability to attract attention. Thus, the light can be white, red, green, blue, yellow or other colors. Further, the light color can be periodically changed from one color to another in addition to having a change in intensity.

FIG. 6 is a sectional view of an eye-fixation device in accordance with the invention depicting an alternative configuration for an electric light source 20. In this configuration, the electric light source includes a fiber-optic element 30 that is passed through the wall of the eyecup 10 so that a light emitting portion of the fiber-optic element is visible within the eyecup. The fiber-optic element 30 can be integral with the eyecup wall or it can be readily removable therefrom by, for example, passing it through an aperture in the eyecup 10. Although the fiber-optic element 30 can be associated with a light source, power supply, control circuit, and switch that are integral to the eyecup as described with device configuration illustrated in FIG. 5, the fiber-optic element 30 can include a flexible, elongate filament 32 that is in communication with a power supply, control circuit, switch, and light source that are separated from the eyecup as shown in FIG. 6. In an exemplary embodiment, the elongate filament 32 can have a length ranging from a few centimeters to a meter or more. In this configuration, the power supply, control circuit, switch, and light source are part of an auxiliary unit 34. This configuration is particularly advantageous when one of or all of the power supply, control circuit, switch and light source are too complex, heavy, or expensive to be carried on or form a part of a perhaps disposable eyecup 10. Further, the auxiliary unit can include a music source, such as a digital audio player, to which earphones 36 can be connected by wire or wirelessly.

FIG. 7 is a sectional view of an eye-fixation device in accordance with the invention depicting yet another light source configuration. In this configuration, the light source includes a chemical light source visible within the eyecup. As illustrated, the chemical light source can be a small “pellet” 38 that is squeezed or flexed to cause two solutions to mix, thereby producing light. The pellet, which can have a diameter slightly greater that that of an aperture in the eyecup 10, can be inserted into the aperture and held in place with a friction or interference fit. Chemical light sources can be provided in different colors as desired.

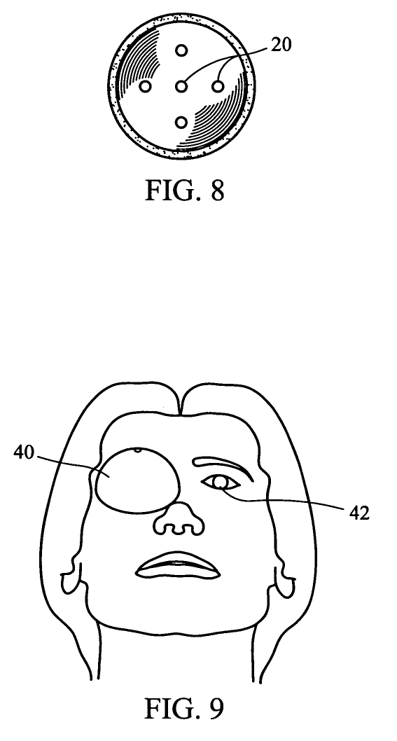

Turning now to FIG. 8, an eye-fixation device is illustrated, wherein the interior of an eyecup 10 is visible. In this configuration, multiple light sources, such as those discussed above, are provided that are visible from within the eyecup 10. Each light source 20 can be individually controlled to draw the eye to the center, left, right, up or down. Although five light sources are shown in FIG. 6, more or less than five lights can be provided as desired to assist in positioning and fixing the eye as is described more fully below. The one or more lights can be positioned other than as shown.

FIG. 9 illustrates an eye-fixation device 40 on a patient's right eye in preparation for treatment of the left eye 42. Thus, in use, a method of reducing motion of an eye during a medical procedure includes the steps of selecting an eye to be treated; placing any eyecup over the eye that is not being treated; and introducing light into the eyecup. The eye covered by the eyecup fixates on the light source; and, as both eyes track together in a person with normal physiology, the uncovered eye that is being treated will also stop moving. In the event that the eye covered by the eyecup begins to move, or to prevent it from moving, the light source intensity and or color can be modulated as described above. If the eye being treated need to be stabilized in different orientations, one or more other light sources (in a device configured as shown in FIG. 8) can be activated.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An eye-fixation device comprising:
an eyecup; and
a source of light within the eyecup.

2. The eye-fixation device of claim 1, further comprising an eyecup fixation element.

3. The eye-fixation device of claim 2, wherein the eyecup fixation element includes an adhesive.

4. The eye-fixation device of claim 2, wherein the eyecup fixation element includes a strap secured to the eyecup.

5. The eye-fixation device of claim 4, wherein a portion of the strap is elastic.

6. The eye-fixation device of claim 2, wherein the light source includes an aperture in the eyecup.

7. The eye-fixation device of claim 6, further comprising a transparent element spanning the aperture.

8. The eye-fixation device of claim 6, further comprising a translucent element spanning the aperture.

9. The eye-fixation device of claim 2, wherein the light source includes a chemical light source visible within the eyecup.

10. The eye-fixation device of claim 2, wherein the light source includes an electric light source visible within the eyecup.

11. The eye-fixation device of claim 10, wherein the electric light source includes a light emitting diode.

12. The eye-fixation device of claim 10, wherein the electric light source includes a fiber-optic element.

13. The eye-fixation device of claim 10, further including a power supply in communication with the electric light source.

14. The eye-fixation device of claim 13, wherein the power supply includes a battery affixed to the eyecup.

15. The eye-fixation device of claim 13, wherein the power supply is separated from the eyecup.

16. The eye-fixation device of claim 13, further comprising a control circuit in communication with the electric light source to control the intensity of the light produced by the electric light source.

17. The eye-fixation device of claim 16, wherein the control circuit is configured to cyclically vary the intensity of the light produced by the electric light source.

18. The eye-fixation device of claim 1, wherein the light source blinks.

19. The eye-fixation device of claim 18, wherein the light source provides more than one color of light.

20. An eye-fixation device comprising:
an eyecup having a soft, compliant lip;
a blinking electric light source visible within the eyecup; and
a battery affixed to the eyecup and in communication with the electric light source.

21. The eye-fixation device of claim 20, further comprising
a control circuit for controlling the intensity of the of electric light source; and
an eyecup fixation element.

22. A method of reducing motion of an eye during a medical procedure comprising:
selecting an eye to be treated;
placing an eyecup over the eye that is not being treated; and
introducing light into the eyecup.
